(19) United States
(12) Patent Application Publication
(10) Pub. No.: US 2025/0297002 A1
(43) Pub. Date: Sep. 25, 2025

(54) BISPECIFIC T-CELL ENGAGER, RECOMBINANT ONCOLYTIC VIRUS THEREOF, AND USE THEREOF

(71) Applicant: SHANGHAI SINOBAY BIOTECHNOLOGY CO., LTD., Shanghai (CN)

(72) Inventors: Jianqing Xu, Shanghai (CN); Xiaoyan Zhang, Shanghai (CN); Xiangqing Ding, Shanghai (CN); Qibin Liao, Shanghai (CN); Shiyu Wang, Shanghai (CN); Junhua Zhou, Shanghai (CN)

(21) Appl. No.: 18/724,346

(22) PCT Filed: Dec. 27, 2021
(86) PCT No.: PCT/CN2021/141634

§ 371 Date: Sep. 24, 2024

(51) Int. Cl. C07K 16/28; A61K 35/768; A61P 35/00; C12N 7/00

(52) U.S. Cl. CPC C07K 16/2803; A61K 35/768; A61P 35/00; C07K 16/2809; C12N 7/00; C07K 2317/31; C07K 2317/622; C07K 2317/73; C07K 2317/92; C12N 2710/24121; C12N 2710/24132; C12N 2710/24151

(57) ABSTRACT

Provided by the present invention are a bispecific T-cell engager, recombinant oncolytic virus thereof, and use thereof. The present invention provides an αCD47 and αCD3 bispecific T-cell engager. The present invention also provides an isolated nucleic acid molecule that encodes said bispecific T-cell engager. The present invention also provides an expression framework of said bispecific T-cell engager BiTE. The present invention also provides a recombinant oncolytic virus, and said oncolytic virus is operably inserted with or contains the expression framework of said bispecific T-cell engager BiTE. In the present invention, the bispecific T-cell engager is combined with the oncolytic virus, and in comparison with pure gene therapy or virotherapy, the oncolytic virus significantly enhances the inhibition capability on malignant tumors.

### TECHNICAL FIELD

The present invention is in the field of biomedical technology and relates to a novel bispecific T-cell engager (BiTE) comprising human CD47 and human CD3 specific binding fragments. The present invention also relates to a recombinant oncolytic virus comprising the bispecific T-cell engager (BiTE). The present invention further provides a method for preparing the bispecific T-cell engager and the oncolytic virus and the use thereof in antitumor.

### BACKGROUND ART

Cancer is the second leading cause of death worldwide, accounting for an estimated 9.6 million deaths in 2018. Globally, approximately one sixth deaths are caused by cancer. The situation in China is particularly worrying, in part because of rapid population growth and socio-economic development. Although the incidence of cancer in China is lower than that in developed European and American countries, the cancer mortality rate in China is 30%-40% higher than that in European and American countries, and the prognosis is significantly worse. Nowadays, cancer is getting more and more attention in China, and the related cancer treatment methods, especially tumor immunotherapy with the help of the human body's immune system, are increasingly becoming the focus of attention.

Human CD47, an integrin-associated protein, is a cell membrane surface glycoprotein belonging to the immunoglobulin superfamily. CD47 is widely expressed on the surface of various cancer cells. It releases the “don't eat me” signal by linking to signaling regulatory protein alpha (SIRPα) on the surface of tumor phagocytes, thereby preventing macrophage phagocytosis. In recent years, many companies have invested a lot of manpower and material resources in the research and development of CD47 antibodies, but the results have been minimal after entering the clinic. Currently, the best level of CD47 antibodies is that they can be included in combination drugs. The main reason for the poor efficacy of the treatment is that the side effects are difficult to be controlled, which is attributed to the lethal weakness of CD47, i.e. expression on erythrocytes. When the CD47 antibody drug or SIRPα-Fc fusion protein binds to red blood cells, it can cause red blood cells to agglutinate and then trigger the rupture of red blood cells; the CD47 antibody drug with Fc of IgG1 can also activate phagocytosis of red blood cells by macrophages or antibody-dependent cell-mediated cytotoxicity (ADCC), further trigger lysis of red blood cells and eventually lead to the occurrence of anemia.

How to maximize the use of CD47 targets in killing tumor cells while reducing the damage to red blood cells has been the key to the successful development of CD47-targeted drugs. The bispecific T-cell engager BiTE represents a class of bispecific antibodies with significant anti-tumor effects, which are capable of target-activating autologous T cells to kill tumor cells. BiTE consists of two single-stranded variable fragments (scFv) connected in series by a flexible linker. One scFv recognizes the T cell surface protein CD3εH, while the other scFv recognizes a specific tumor cell surface antigen. This structure of BiTE and the ability to specifically bind proteins allows it to physically bridge T cells to tumor cells to form T cell-BiTE-tumor cell complexes, induce immune synapse formation, stimulate T cell activation, and produce tumor-killing cytokines. In recent years, BiTE has made remarkable progress in anti-tumor research and achieved an ideal therapeutic effect in clinical practice.

Tumor-targeted oncolytic viruses have attracted considerable attention over the last decades due to the limited therapeutic efficacy and severe side effects of standard tumor treatment regimens in the treatment of advanced tumors. Oncolytic viruses are increasingly becoming the first choice for anti-tumor because of their ability to selectively infect and lyse tumor cells locally. The tumor selectivity of an oncolytic virus may be a result of its own tumor tropism or genetic modification. Oncolytic viruses can act on multiple cellular pathways, thereby reducing tumor resistance and also inducing different forms of cell death. In addition, oncolytic viruses can break the immune tolerance of the tumor microenvironment and induce long-term tumor-specific immune responses. Oncolytic viruses can specifically transport therapeutic proteins into tumor tissue with increased expression levels in malignant tumor cells following further viral replication. In addition, oncolytic viruses can be used in combination with chemotherapy, radiotherapy, and immunotherapy.

In 2006, the oncolytic adenovirus product (oncorine) was used for the clinical treatment of nasopharyngeal carcinoma in China. The E1B-55 kD gene of human adenovirus type 5 is deleted in this oncolytic virus, which could replicate and proliferate in the cancer cells mutated in the p53 gene and kill the host cells, resulting in the oncolytic therapeutic effect; simultaneous deletion of the E3 region allows the tumor antigen message to activate T cell immunity via dendritic cell transmission. However, clinical data show that oncolytic virus oncorine combined with chemotherapy is less effective than radiotherapy in patients with nasopharyngeal carcinoma.

In 2015, Amgen's oncolytic herpes simplex virus (talimogene laherparepvec, T-VEC) was approved by the US FDA for the treatment of melanoma. In December of the same year, it was approved by the European Union for the local treatment of unresectable skin, subcutaneous, and lymph node lesions in patients with melanoma who relapsed after the first surgery. The results of the T-VEC clinical study have greatly promoted the development of the oncolytic virus in the field of tumor therapy. However, the intratumoral administration method limits the types of treatments and can only be used for tumors close to the body surface that are easy to operate on. There are problems of difficulty in administering drugs and incomplete treatment for many non-superficial solid tumors and metastatic tumors. If it can be confirmed that intravenous administration still has a good tumor treatment effect, its clinical application value will be greatly improved; due to the limited oncolytic effects of herpes viruses themselves, incomplete clearance of bulky and/or metastatic tumors, there is a need to combine other therapeutic approaches to enhance anti-tumor effects.

The biological characteristics and pathogenesis of the vaccinia virus (VV) are relatively clear, which plays a key role in the elimination of smallpox, and its safety in the human body has been fully demonstrated. According to the characteristics of pathogenicity and host range, the vaccinia virus can be divided into the WR (Western reserve) strain, Wyeth strain, Copenhagen strain, Lister strain, and Tian Tan strain. Vaccinia virus is used as a vector for multiple recombinant vaccines, such as influenza virus and human immunodeficiency virus, because of its wide host range, high conservation, good safety, and large capacity of exogenous genes. As for the development of oncolytic viruses, most of them are in the preclinical research stage, and only a few of them are in the clinical research stage.

The fastest-progressing research on using vaccinia virus as an oncolytic virus to treat tumors is Pexa-Vec (JX-594) developed by Jennerex, USA. JX-594 is based on the Wyeth strain virus, inserts hGM-CSF and LacZ genes in the TK region, and due to deletion of the thymokinase gene, JX-594 can express and replicate in cancer cells expressing thymokinase at a high level but has no effect on normal cells. At the same time, JX-594 can express GM-CSF in tumor cells by inserting the GM-CSF gene, activating the body's anti-tumor immune response. The clinical trial results of JX-594 in multiple tumor types showed that intratumoral administration or intravenous drip administration had good tolerability, and the effect of Pexa-Vec combined with sorafenib was better than that of a single drug group. The results of the interim analysis showed that it was not likely to prolong the survival of patients. The Phase III clinical trial of Pexa-Vec, which was originally planned to be launched in 2020, was terminated ahead of schedule.

The oncolytic virus (GL-ONC1, also known as GLV-1h68) developed by Genelux, USA, is based on vaccinia virus (Lister strain), deleting its genes F14.5L, TK (encoding thymidine kinase) and HA (encoding hemagglutinin) to enhance tumor targeting, and inserting luciferase-GFP fusion protein, β-galactosyl transferase and β-glucuronidase, respectively, for poxvirus screening and production preparation. The phase I clinical trial of intravenous administration of GL-ONC1 has been completed and showed good safety and efficacy, without dose-limiting toxicity and reaching the maximum tolerated dose, and all patients had neutralization reaction to GL-ONC1; GL-ONC1 is currently undergoing an expanded trial with intravenous administration.

The life cycle of the vaccinia virus is strictly carried out in the cytoplasm of the host cell, and the thymidine kinase gene of the vaccinia virus is required for successful replication of the genome of the progeny virus. However, in the normal tissue cell cycle, the synthesis of cellular Thymidine Kinase (TK) occurs in the S phase of the cell division cycle, after cell division is completed, thymidine kinase is degraded inside the cell, so the concentration of thymidine kinase in the cytoplasm is low. However, the cell division of tumor cells is active, and thymidine kinase is continuously synthesized. Using this feature, the thymidine kinase gene of the vaccinia virus is deleted, resulting in the specific amplification of the vaccinia virus in tumor cells and oncolysis.

Evidence from clinical trials indicates that the vaccinia virus has shown some initial advantages in tumor therapy. Most of the existing designs using vaccinia virus as an oncolytic virus have high safety and clinical efficacy to be further observed, but further optimization is needed in immunomodulation and precise tumor targeting.

Based on the above, there is no bispecific T-cell engager in the prior art that is particularly suitable for recombinant oncolytic viruses, and there is currently a need for safer, more targeted bispecific T-cell engagers and oncolytic viruses comprising them.

### SUMMARY OF THE INVENTION

It is therefore an object of the present invention to address the deficiencies of the prior art to provide a bispecific T-cell engager. The present invention also provides recombinant oncolytic viruses expressing the bispecific T-cell engager (BiTE). The bispecific T-cell engager (BiTE) provided by the present invention overcomes the limitation of solid tumor cells to human T cells in tumor immune editing (immunosuppression and immune evasion), and can directly recruit human T cells to the interior of solid tumors for tumor cell killing, with broad application prospects.

The objects of the present invention are achieved by the following technical solutions:

In one aspect, the invention provides an αCD47 and αCD3 bispecific T-cell engager αCD47-αCD3

BiTE, comprising a fusion protein of any one of the following formulae:

VL_{αCD47}-L-VH_{αCD3}-L-VL_{αCD3}-L-VH_{αCD47};

or

VH_{αCD47}-L-VH_{αCD3}-L-VL_{αCD3}-L-VL_{αCD47}

wherein the VH_{αCD47} is the heavy chain variable region of a CD47 antibody comprising the following three complementary determining regions:
(i) VH CDR1 consisting of the following sequence: SEQ ID NO: 1, or a sequence having one or several amino acid substitutions, deletions, or additions compared thereto,
(ii) VH CDR2 consisting of the following sequence: SEQ ID NO: 2, or a sequence having one or several amino acid substitutions, deletions, or additions compared thereto, and
(iii) VH CDR3 consisting of the following sequence: SEQ ID NO: 3, or a sequence having one or several amino acid substitutions, deletions, or additions compared thereto;
preferably, the substitution of any one of (i)-(iii) is a conservative substitution;
preferably, the VH_{αCD47} comprises VH CDR1 as shown in SEQ ID NO: 1, VH CDR2 as shown in SEQ ID NO: 2, VH CDR3 as shown in SEQ ID NO: 3;
wherein the VL_{αCD47} is the light chain variable region of a CD47 antibody comprising the following three complementary determining regions:
(iv) VL CDR1 consisting of the following sequence: a sequence as shown in any one of SEQ ID NO: 4, SEQ ID NO: 7, SEQ ID NO: 10, SEQ ID NO: 13 or SEQ ID NO: 16, or having one or several amino acid substitutions, deletions, or additions compared thereto,
(v) VL CDR2 consisting of the following sequence: a sequence as shown in any one of SEQ ID NO: 5, SEQ ID NO: 8, SEQ ID NO: 11, SEQ ID NO: 14, or SEQ ID NO: 17, or having one or several amino acid substitutions, deletions, or additions compared thereto, and
(vi) VL CDR3 consisting of the following sequence: a sequence as shown in any one of SEQ ID NO: 6, SEQ ID NO: 9, SEQ ID NO: 12, SEQ ID NO: 15, or SEQ ID NO: 18, or having one or several amino acid substitutions, deletions, or additions compared thereto;
preferably, the substitution of any one of (iv)-(vi) is a conservative substitution;
preferably, the VL_{αCD47} comprises VL CDR1 as shown in SEQ ID NO: 4, VL CDR2 as shown in SEQ ID NO: 5, and VL CDR3 as shown in SEQ ID NO: 6;
or the VL_{αCD47} comprises VL CDR1 as shown in SEQ ID NO: 7, VL CDR2 as shown in SEQ ID NO: 8, and VL CDR3 as shown in SEQ ID NO: 9;
or the VL_{αCD47} comprises VL CDR1 as shown in SEQ ID NO: 10, VL CDR2 as shown in SEQ ID NO: 11, and VL CDR3 as shown in SEQ ID NO: 12;
or the VL_{αCD47} comprises VL CDR1 as shown in SEQ ID NO: 13, VL CDR2 as shown in SEQ ID NO: 14, and VL CDR3 as shown in SEQ ID NO: 15;
or the VL_{αCD47} comprises VL CDR1 as shown in SEQ ID NO: 16, VL CDR2 as shown in SEQ ID NO: 17, and VL CDR3 as shown in SEQ ID NO: 18.

In the bispecific T-cell engager according to the invention, the VH_{αCD47} comprises three CDRs contained in the heavy chain variable region as shown in SEQ ID NO: 19;

preferably, the VL_{αCD47} comprises three CDRs contained in the light chain variable region as shown in any one of SEQ ID NOs: 20-24; and

preferably, the three CDRs contained in the heavy chain variable region and/or the three CDRs contained in the light chain variable region are defined by the Kabat, Chothia, or IMGT numbering system.

In the bispecific T-cell engager according to the invention, the VH_{αCD47} comprises an amino acid sequence selected from the group consisting of:
(i) a sequence as shown in SEQ ID NO: 19;
(ii) a sequence having one or several amino acid substitutions, deletions, or additions compared to the sequence as shown in SEQ ID NO: 19; or
(iii) a sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as shown in SEQ ID NO: 19;
and/or,
the VL_{αCD47} comprises an amino acid sequence selected from the group consisting of:
(iv) a sequence as shown in any one of SEQ ID NOs: 20-24;
(v) a sequence having one or several amino acid substitutions, deletions, or additions compared to the sequence as shown in any one of SEQ ID NOs: 20-24; or
(vi) a sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as shown in any one of SEQ ID NOs: 20-24; and
preferably, the substitution in (ii) or (v) is a conservative substitution.

In the bispecific T-cell engager according to the invention, the VH_{αCD3} comprises an amino acid sequence selected from the group consisting of:
(i) a sequence as shown in SEQ ID NO: 25 or 27;
(ii) a sequence having one or several amino acid substitutions, deletions, or additions compared to the sequence as shown in SEQ ID NO: 25 or 27; or
(iii) a sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as shown in SEQ ID NO: 25 or 27;
and/or,
the VL_{αCD3} comprises an amino acid sequence selected from the group consisting of:
(iv) a sequence as shown in SEQ ID NO: 26 or 28;
(v) a sequence having one or several amino acid substitutions, deletions, or additions compared to the sequence as shown in SEQ ID NO: 26 or 28; or
(vi) a sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as shown in SEQ ID NO: 26 or 28; and
preferably, the substitution in (ii) or (v) is a conservative substitution.

In the bispecific T-cell engager according to the invention, the VH_{αCD47}, VH_{αCD3}, VL_{αCD47}, and VL_{αCD3} are linked by a linker peptide;

preferably, the VH_{αCD47}, VH_{αCD3}, VL_{αCD47}, and VL_{αCD3} are optionally linked by one, two, or three linker peptides;

preferably, the bispecific T-cell engager comprises a fusion protein according to any one of the following formulae:

VL_{αCD47}-L-VH_{αCD3}-L-VL_{αCD3}-L-VH_{αCD47};

or

VH_{αCD47}-L-VH_{αCD3}-L-VL_{αCD3}-L-VL_{αCD47}
more preferably, L is KESGSVSSEQLAQFRSLD (SEQ ID NO: 45), EGKSSGSGSESKST (SEQ ID NO: 46), GGGGGG (SEQ ID NO: 47), GGGGGGGG (SEQ ID NO: 48) or (GGGGS) n (SEQ ID NO: 49); further preferably, n is an integer from 1 to 5, most preferably n is 3; and
preferably, the bispecific T-cell engager comprises a fusion protein of the following formula:

VH_{αCD47}-L-VH_{αCD3}-L-VL_{αCD3}-L-VL_{αCD47}
wherein the VH_{αCD47} is as shown in SEQ ID NO: 19, the VL_{αCD47} is as shown in SEQ ID NO: 21 or 24, the VH_{αCD3} is as shown in SEQ ID NO: 25, VL_{αCD3} is as shown in SEQ ID NO: 26, and the L is GGGGSGGGGSGGGGS (SEQ ID NO: 50).

The present invention also provides an isolated nucleic acid molecule that encodes the bispecific T-cell engager;
preferably, the nucleic acid molecule comprises a nucleic acid sequence as shown in any one of SEQ ID NOs: 31-35, the nucleic acid sequence is the coding sequence for VL_{αCD47}; and
preferably, the nucleic acid molecule comprises a nucleic acid sequence as shown in SEQ ID NO: 36, the nucleic acid sequence is the coding sequence for VH_{αCD47}.

An expression framework for the bispecific T-cell engager according to the invention:
5′-E1-E2-E3-E4-E5-E6-E7-3′
wherein:
E1 is the CMV enhancer or/and other cis-acting elements, preferably comprises the nucleic acid sequence as shown in SEQ ID NO: 37;
E2 is a recombinantly expressed promoter, preferably a CMV promoter, more preferably comprising the nucleic acid sequence as shown in SEQ ID NO: 38;
E3 is a 5′ untranslated region, optionally comprising no or one intron sequence, optionally comprising one or more restriction enzyme sites, preferably comprises the nucleic acid sequence as shown in SEQ ID NO: 39;
E4 is a coding nucleotide sequence of the signal peptide of the BiTE protein; preferably, the signal peptide is derived from a human or mouse signal peptide; preferably E4 comprises the nucleic acid sequence as shown in SEQ ID NO: 40;
E5 is the coding nucleotide sequence of the bispecific T-cell engager;
E6 is a 3′ untranslated region, optionally comprising one or more restriction enzyme sites preferably comprises the nucleic acid sequence as shown in SEQ ID NO: 41; and
E7 is the SV40 transcription termination signal region, preferably comprising the nucleic acid sequence as shown in SEQ ID NO: 42.

In yet another aspect, the present invention provides a recombinant oncolytic virus, and the oncolytic virus is operably inserted with or contains the expression framework of the bispecific T-cell engager BiTE.

Preferably, the expression framework is located in the thymidine kinase (TK) region of the recombinant oncolytic virus.

Preferably, the expression framework can be expressed alone, and can also be fusion expressed with other genes or fragments.

Preferably, the recombinant oncolytic virus further comprises gene coding sequences for other immunomodulatory factors, more preferably, the other immunomodulatory factors include but are not limited to IL-1, IL-2, IL-3, IL-7, IL-11, IL-12, IL-15, IL-17, IL-18, IL-21, IL-33, IL-35, IL-37, GM-CSF, IFN-α, IFN-β, IFN-γ, anti-PD-1/PD-L1 antibody, anti-CTLA-4 antibody, anti-Lag-3 antibody, anti-TIGIT antibody or anti-Tim-3 antibody; or the recombinant oncolytic virus further comprises gene coding sequences of apoptosis- and pyroptosis-related proteins, including but not limited to apoptosis-related factor 1 (Apaf-1), interleukin-1β converting enzyme (ICE), Bcl-2 protein, Fas/APO-1, p53, myc, ataxia telangiectasia mutant gene (ATM), gasdermin D, gasdermin E, etc.; or the recombinant oncolytic virus further comprises a small RNA targeting immunomodulatory genes, apoptosis and pyroptosis genes.

In the recombinant oncolytic virus according to the present invention, the viral backbone of the oncolytic virus is derived from a modified or engineered vaccinia virus Tian Tan strain, vaccinia virus New York strain, vaccinia virus Copenhagen strain, vaccinia virus canary strain, vaccinia virus Ankara strain, adenovirus, adeno-associated virus, herpes simplex virus, varicella-zoster virus (VZV), respiratory syncytial virus (RSV), Semliki forest virus (SFV), EB virus, cytomegalovirus, human Herpesvirus type 6, smallpox virus, vaccinia virus, molluscum contagiosum virus, sheep aphthovirus, reovirus, rotavirus, enterovirus, Seneca virus, poliovirus, coxsackie virus, rhinovirus, hepatitis A virus, foot and mouth disease virus, togavirus, alphavirus, Semiliki forest virus, eastern equine encephalitis virus, Sindbis virus, rubella virus, coronavirus, flavivirus, hepatitis C virus, Japanese encephalitis virus, St. Louis encephalitis virus, Murray Valley fever virus, yellow fever virus, West Nile virus, zika virus, dengue virus, Ebola virus, Marburg virus, arenavirus, Lassa fever virus, lymphocytic choriomeningitis virus, Pichinde virus, Junin virus, Machupo virus, Hantavirus, Rift Valley fever virus, Paramyxovirus, human parainfluenza virus, Mumps virus, simian virus 5, measles virus, vesicular stomatitis virus, rabies virus, orthomyxovirus, influenza A virus, influenza B virus, influenza C virus, hepatitis D virus, simian immunodeficiency virus, human immunodeficiency virus type 1 and human immunodeficiency virus type 2, Rous sarcoma virus, human T-cell leukemia virus type 1, simian foamy virus, hepatitis B virus, hepatitis E virus, human papilloma virus, or polyomavirus.

Preferably, the oncolytic viral backbone is an intracellular maturation virus, an intracellular packaging virus, a cell-associated packaging virus, or an extracellular packaging virus.

The recombinant oncolytic virus according to the present invention is a recombinant vaccinia virus Tian Tan strain comprising a gene encoding the bispecific T-cell engager αCD47-αCD3-BITE, named rTV-αCD47-αCD3-BITE, with a deposit accession number of CCTCC NO: V202081, a deposit date of Jan. 2, 2021, and a depositary institution of China Center for Type Culture Collection.

In yet another aspect, the present invention provides a method for preparing the recombinant oncolytic virus, comprising the steps of:
1) synthesizing an expression framework of the bispecific T-cell engager BiTE;
2) subcloning the expression framework obtained in step 1) into a shuttle plasmid of an oncolytic virus to construct a recombinant plasmid vector;
3) transfecting the recombinant plasmid vector obtained in step 2) into an oncolytic virus, and creening the same so as to obtain a recombinant oncolytic virus;
optionally, culturing the obtained recombinant oncolytic virus.

In a specific embodiment, the present invention provides a method for preparing a recombinant vaccinia virus Tian Tan strain, comprising the steps of:
1) synthesizing an expression framework of the bispecific T-cell engager αCD47-αCD3-BITE, which comprises the nucleic acid sequence as shown in any one of SEQ ID NOs: 31-42;
2) subcloning the synthesized expression framework into the TK region of the vaccinia virus shuttle plasmid (pSC65) to construct the recombinant plasmid pSC65-αCD47-αCD3-BITE;
3) transfecting the pSC65-αCD47-αCD3-BITE plasmid into TK143-cells which have been infected with wild-type vaccinia virus by means of gene homologous recombination, and homologously recombing the two to produce recombinant vaccinia virus rTV-αCD47-αCD3-BITE; screening the same to obtain a recombinant oncolytic vaccinia virus wherein the TK region comprises the coding sequence of αCD47-αCD3-BITE;
wherein, the expression framework of the bispecific T-cell engager αCD47-αCD3-BITE is controlled by the early/late promoter p7.5 of vaccinia virus.

Preferably, amplifying the recombinant vaccinia virus using VERO cells comprises the specific steps of: replacing the medium with the low-concentration fetal bovine serum maintenance medium when VERO cells are cultured to a density close to 100%, adding oncolytic vaccinia virus (the inoculation amount is about 0.02 MOI per 10 cm culture plate), and placing the same into an incubator for culture, collecting the virus solution after completion of amplification of recombinant poxvirus, freeze-thawing the same repeatedly, and then purifying the same by density gradient centrifugation using sucrose solution.

In a further aspect, the present invention also provides use of the bispecific T-cell engager or recombinant oncolytic virus for the preparation of an anti-tumour medicament; wherein the tumor is selected from the group consisting of B-cell lymphoma; T-cell lymphoma; melanoma; prostate cancer; renal cell carcinoma; sarcoma; glioma, such as high-grade glioma; blastoma, such as neuroblastoma; osteosarcoma; plasmacytoma; histiocytoma; pancreatic cancer; breast cancer; lung cancer, such as small cell lung cancer and non-small cell lung cancer; gastric cancer; liver cancer; colon cancer; rectal cancer; esophageal cancer; colorectal cancer; hematopoietic cancer; testicular cancer; cervical cancer; ovarian cancer; bladder cancer; squamous cell cancer; adenocarcinoma; AIDS-related lymphoma; bladder cancer; brain cancer; nervous system cancer; head and neck cancer; head and neck squamous cell carcinoma; Hodgkin's lymphoma; non-Hodgkin's lymphoma; or hematological tumorigenesis disease.

In another aspect, the invention provides a method of treating a tumor, comprising administering to a subject in need thereof a therapeutically effective amount of a bispecific T-cell engager or a recombinant oncolytic virus. The method according to the present invention further comprises administering to the subject in need thereof an additional chemotherapeutic agent, a radiation therapy technique, a surgical treatment, an immune cell drug (including but not limited to CAR-T, NK, NKT, INKT, CAR-NK, CAR-NKT, CAR-iNKT, etc.), other oncolytic viruses; preferably, the method is intravenous injection or intratumoral injection.

The inventive concept of the present invention is as follows: The present invention provides a novel αCD47-αCD3-BITE bispecific T-cell engager with good anti-tumor activity in vitro. Further, the present inventors have provided a vaccinia virus Tian Tan strain carrying the bispecific T-cell engager of the present invention on the basis of a research mode of vaccinia virus Tian Tan strain. Then, the inventors of the present invention found that the vaccinia virus Tian Tan strain itself has a strong anti-tumor effect; the bispecific T-cell engager carrying αCD47-αCD3-BITE can avoid the binding of CD47 antibody to the red blood cells of peripheral blood, recognize tumor cells by using the broad-spectrum expression characteristics of CD47, and activate T cells by CD3 antibody, thereby playing the role of killing tumor cells, improving the effect of tumor immunotherapy and increasing safety.

Advantageous effects of the present invention are:
1. The bispecific T-cell engager (BiTE) provided by the present invention has a small molecular weight, so it is capable of penetrating the intercellular space to reach the tumor microenvironment (TME) inside solid tumors, thereby being capable of efficiently mediating the killing of cancer cells by T cells.
2. The present invention combines a bispecific T-cell engager with an oncolytic virus to provide a vaccinia virus Tian Tan strain highly expressing the bispecific T-cell engager αCD47-αCD3-BITE gene. When the oncolytic virus is administered to tumor lesions, it lyses tumor cells to exert an oncolytic effect, and at the same time carries a tumor-targeted gene high-killing antibody against human CD47, blocks the binding of CD47 to SIRPα, and promotes macrophage phagocytosis in vivo, so as to enhance the anti-tumor effect. The oncolytic virus significantly enhances the ability to inhibit malignant tumors when compared to gene therapy or virotherapy alone.
3. According to the in vitro experimental study of the present invention, it is proved that the vaccinia virus Tian Tan strain of the present invention can treat liver cancer and malignant lung cancer, achieve good targeting to tumors and anti-tumor effect, have a relatively complete virus amplification and quality control system, and lay the foundation for further industrialization, and therefore, the present invention has a good application prospect.

### BRIEF DESCRIPTION OF THE DRAWINGS

Hereinafter, embodiments of the present invention will be described in detail with reference to the accompanying drawings, in which:

FIG. 1 shows the construction of the shuttle plasmid vector αCD47-αCD3 BiTE hlgG1 WT of the present invention and the expression and purification of five corresponding BiTE proteins; wherein, FIG. 1A is an expression map of shuttle plasmid hlgG1 WT integrated with the αCD47-αCD3-BITE expression framework; FIG. 1B is the result of supernatants of medium with 293T transiently expressing αCD47-αCD3-BITE after protein purification using a nickel column.

FIG. 2 shows the effect of αCD47-αCD3-BITE mediated T cells in vitro against human ovarian adenocarcinoma cells; wherein, compared with the T cell control group, the BiTE composed of the CD47 antibody of clone No. 68 and αCD3 can well mediate the killing of SK-OV3 by T cells. The results show that αCD47-αCD3-BITE can recognize CD47 tumor cells and activate T cells, thereby mediating the specific killing of the tumor cells by T cells; it has been shown that a bispecific T-cell engager (BITE) comprising specific binding of human CD47 and human CD3 mediates T cell antitumor activity well in vitro and has a broad spectrum of properties.

FIG. 3 shows the effect of αCD47-αCD3-BITE mediated T cells in vitro against lung cancer cells; wherein, compared with the T cell control group, BiTE composed of CD47 antibody of clone No. 101 and αCD3 can well mediate the killing of NCI-H292 tumor cells by T cells. Compared with the T cell alone group, the BiTE group has an obvious killing effect on NCI-H292 cells; it has been shown that a bispecific T-cell engager (BITE) comprising specific binding of human CD47 and human CD3 mediates T cell antitumor activity well in vitro and has a broad spectrum of properties.

FIG. 4 shows the construction of the vaccinia virus Tian Tan strain shuttle plasmid vector (αCD47-αCD3-BITE) and the expression of αCD47-αCD3-BITE protein. FIG. 4A is an expression map of vaccinia virus shuttle plasmid pSC65 integrated with αCD47-αCD3-BITE gene; FIG. 4B is the results of PCR identification of the insert fragment αCD47-αCD3-BITE in the recombinant vaccinia virus TV-αCD47-αCD3-BITE; FIG. 4C shows CD47-αCD3-BITE protein expression in supernatants of recombinant vaccinia virus-infected VERO cells. As can be seen from FIG. 4, αCD47-αCD3-BITE carried in the recombinant vaccinia virus rTV-αCD47-αCD3-BITE was successfully expressed.

FIG. 5 shows that the αCD47-αCD3-BITE carried by the recombinant vaccinia virus rTV-αCD47-αCD3-BITE has a high affinity for the CD47 protein; FIG. 5 is the flow cytometry results of affinity assay of cell supernatant collected from VERO cells infected with recombinant vaccinia virus rTV-αCD47-αCD3-BITE with lung cancer cells A549 expressing human CD47; the results show that compared with wild-type vaccinia virus, the protein expressed by the recombinant vaccinia virus rTV-αCD47-αCD3-BITE has more than 85% binding rate to CD47.

FIG. 6 shows the killing effect of recombinant vaccinia virus of vaccinia virus Tian Tan strain rTV-αCD47-αCD3-BITE on A549 cells in vitro. The experiment was divided into four groups, including a wild vaccinia virus-infected supernatant control group, a wild vaccinia virus-infected supernatant+T cell group, a recombinant vaccinia virus-infected supernatant control group, and a recombinant vaccinia virus-infected supernatant+T cell group. It can be seen from the figure that compared with the blank control group, the recombinant vaccinia virus-infected supernatant+T cell group has a significant killing effect on lung cancer cells A549 cells.

FIG. 7 shows the anticancer effects of αCD47-αCD3-BITE proteins of different structures.

### DEPOSIT INFORMATION

The vaccinia virus Tian Tan strain is named rTV-αCD47-αCD3-BITE and has the deposit accession number of CCTCC NO: V202081, the deposit date of Jan. 2, 2021, and the depositary institution of China Center for Type Culture Collection (address: Wuhan University, Wuhan, China).

### Mode of Carrying Out the Invention

### Example 1: Construction and Expression Verification of 293T Recombinantly Expressed Plasmid of αCD47-αCD3-BITE

1.1 Construction of hIgG1 WT vector with αCD47-αCD3-BITE target gene

First, five high affinity anti-human CD47 antibodies were screened, their heavy chain and light chain variable regions were selected, and a fusion protein was constructed according to the following formula:

VH_{αCD47}-L-VH_{αCD3}-L-VL_{αCD3}-L-VL_{αCD47};

wherein the VH_{αCD47} was as shown in SEQ ID NO: 19, the VL_{αCD47} was as shown in any one of SEQ ID NOs: 20-24, the VH_{αCD3} was as shown in SEQ ID NO: 25, VL_{αCD3} was as shown in SEQ ID NO: 26, and the L was GGGGSGGGGSGGGGS.

Also, depending on VL_{αCD47}, Hu004-67 denoted an αCD47-αCD3 BiTE including VL_{αCD47} as shown in SEQ ID NO: 20; Hu004-68 denoted αCD47-αCD3 BiTE comprising VL_{αCD47} as shown in SEQ ID NO: 21; Hu004-73 denoted αCD47-αCD3 BiTE comprising VL_{αCD47} as shown in SEQ ID NO: 22; Hu004-100 denoted αCD47-αCD3 BiTE comprising VL_{αCD47} as shown in SEQ ID NO: 23; Hu004-101 denoted αCD47-αCD3 BiTE comprising VL_{αCD47} as shown in SEQ ID NO: 24, wherein WT Cloning vector AbVec-hIgG1 (GenBank: FJ475055.1).

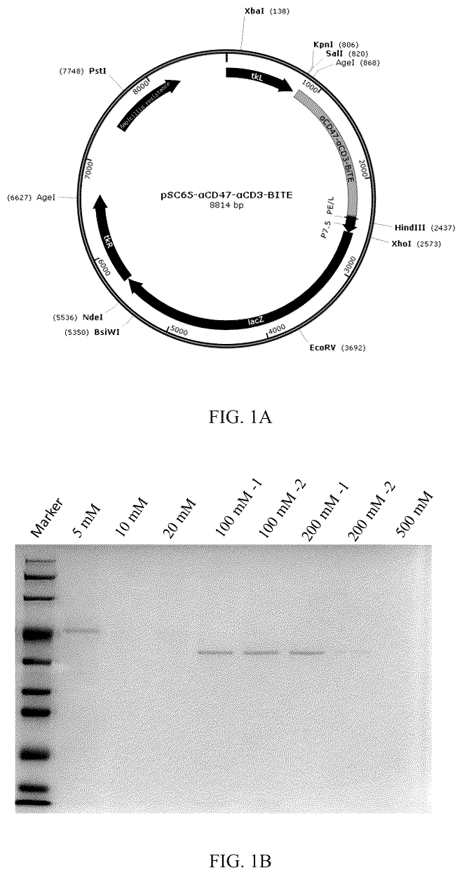

The DNA sequence of αCD47-αCD3-BITE was artificially synthesized according to the structural formula, and the plasmid construct map was shown in FIG. 1a, wherein the coding sequence for VL_{αCD47} was shown in any one of SEQ ID NOs: 31-35, and the coding sequence for VH_{αCD47} was shown in SEQ ID NO: 36.

The synthetic hlgG1 WT vector containing the target gene αCD47-αCD3-BITE was transformed into E. coli TOP10 (Weidi, Cat. No. DL1010S) and grown overnight on plates containing ampicillin. On day 2, single colonies were randomly picked for overnight culture at 37° C. and plasmids were extracted for 293T cell transfection using a plasmid extraction kit (Qiagen, Cat. No. 27106).

1.2 Expression purification of 293T cells with αCD47-αCD3-BITE target gene
1. 293T cell preparation: the supernatant of the cells growing in T175 culture flask with the confluence exceeding 90% was removed, 2 ml of 2.5% Trypsin-EDTA (GIBCO, Cat. No. 25200072) was added to digest the cells until the cells were completely detached, 10 ml of complete medium (DMEM medium+10% FBS+1% PS) was added to stop the digestion, the mixture was centrifuged at 150 g for 5 min to collect the cells, the supernatant was discarded, and the cells were reselected with proper amount of complete medium to make the cell concentration about 8×10⁶˜10×10⁶ cells/ml. The cell suspension was counted and 1.3×10⁷ cells were plated in a 150 mm cell culture dish (Thermo Scientific, Cat. No. 150468) with a medium volume of 25 mL and cultured until the next day for subsequent plasmid transfection.
2. Transfecting 293T cells by hlgG1 WT shuttle plasmid of αCD47-αCD3-BITE: 1 mL DMEM culture medium was taken and put into an EP tube, 20 μg of the hlgG1 WT shuttle plasmid Cloning vector AbVec-hlgG1 (GenBank: FJ475055.1) of αCD47-αCD3-BITE was added to mix well, and then 30 μl FectoPro transfection reagent (Polyplus, Cat. No. 116-001) was added, after that, the mixture was placed on a vortex oscillator immediately and shake vigorously for 15 s, and let it stand at room temperature for 20 min. The 293T cells to be transfected were taken out from the incubator, 10 mL of culture medium was sucked and discarded, the transfection reagent after standing was added while shaking, and the mixture was placed in an incubator containing 5% CO₂ at 37° C. for 4-6 h. The medium was completely removed and Expi293 expression medium (GIBCO, Cat. No. A1435102) was added and incubated at 37° C. in an incubator containing 5% CO₂ for 4 days.
3. Collecting the αCD47-αCD3-BITE expression supernatant: medium was harvested from the expression dishes into a 50 mL centrifuge tube and centrifuged at 4000 g for 5 min and the expression supernatant was transferred to a new 50 mL centrifuge tube.
4. Subjecting the expression supernatant to His tag protein purification: purification was performed using a gravity chromatography column packed with Ni-NTA Agarose (QIAGEN, Cat. No. 30210); after the completion of column loading, the non-specifically adsorbed proteins were eluted with 5 mM and 10 mM imidazole (dissolved in 50 mM NaH₂PO₄: 2H₂O, pH 8.0, 300 mM NaCl) at natural flow rate, and the filtrate was collected; the target protein was then eluted with 20 mM, 100 mM (collecting two tubes in sequence), 200 mM (collecting two tubes in sequence), 500 mM imidazole (dissolved in 50 mM NaH₂PO₄: 2H₂O, pH 8.0, 300 mM NaCl) and the corresponding filtrate was collected.
5. 20 μL of the filtrate was taken to prepare samples and 10% SDS-PAGE detection was performed. As shown in the figure, samples were loaded sequentially from 5 mM imidazole eluate to 500 mM imidazole eluate. The protein of interest was present in the 100 mM eluate and 200 mM shown in FIG. 1B, with a band size of around 54 KD.

### Example 2: Effect of αCD47-αCD3-BITE Mediated T Cells In Vitro Against Human Ovarian Adenocarcinoma Cells

1. SK-OV3 cell plating: SK-OV3 (deposited in Shanghai Sinobay Biotech Co. Ltd.) was a Luciferase-expressing cell line. Cells were plated in 96-well plates at a density of 1×10⁴ cells/well and incubated overnight at 37° C. for attachment.
2. After 24 h, the medium was removed, and seven groups were set, and T cells, B6H12-αCD3-BITE (heavy chain variable region and light chain variable region of B6H12 were shown in SEQ ID NO: 29 and 30, respectively, and the remaining structures were the same as those of the BITE of Example 1 of the present invention) and T cells, Hu004-67-αCD3-BITE and T cells, Hu004-68-αCD3-BITE and T cells, Hu004-73-αCD3-BITE and T cells, Hu004-100-αCD3-BITE and T cells, Hu004-101-αCD3-BITE and T cells were added, respectively.
3. Overnight culture was performed at 37° C. for killing. After 24 h, the supernatant was removed and 50 μL of cell lysate Luciferase cell lysate (Promega Cat. No. E1531) was added per well and incubated for 30 min at room temperature with shaking and 30 μL of Luciferase substrate (Promega, Cat. No. E151A) was added per well. On-machine detection (GloMax Navigator Microplate Luminometer, Promega, Steady-Glo protocol) was conducted.

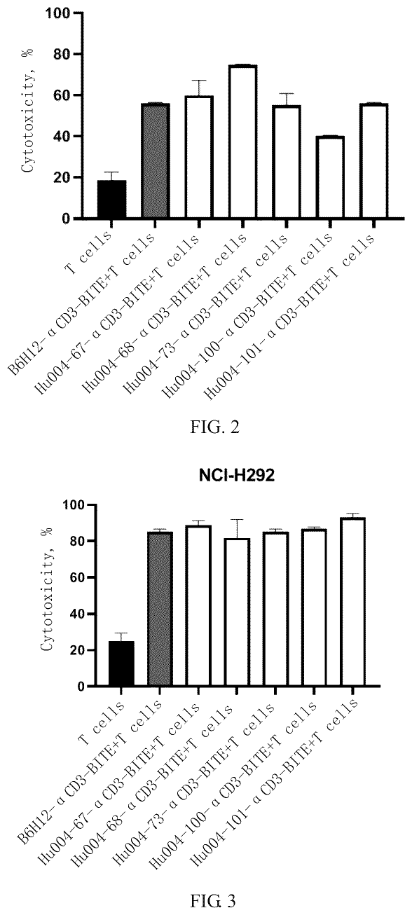

The detected results were shown in FIG. 2. B6H12 was a known CD47 antibody clone (U.S. Pat. No. 9,017,675B2) as a positive control. Hu004-68 and Hu004-101 were selected antibody clones with high affinity for CD47 protein (obtained by antibody screening under entrusted to Osaka Biotech). As can be seen from FIG. 2, against this tumor cell line, the CD47 antibody (scFv)-αCD3-BITE of clone No. 68 showed a 3.5-fold increase in tumor killing activity compared with the T cell control group, a 2-fold increase compared with clone No. 100 with low killing ability, and a 1.5-fold increase compared with the positive control antibody, which can well mediate the in vitro anti-ovarian adenocarcinoma cell effect of T cells.

### Example 3: Effect of αCD47-αCD3-BITE Mediated T Cells In Vitro Against Human Lung Cancer Cells

1. NCI-H292 cell plating: NCI-H292 (deposited in Shanghai Sinobay Biotech Co. Ltd.) was a Luciferase-expressing cell line. Cells were plated in 96-well plates at a density of 1×10⁴ cells/well and incubated overnight at 37° C. for attachment.
2. After 24 h, the medium was removed, and seven groups were set, and T cells, B6H12-αCD3-BITE and T cells, Hu004-67-αCD3-BITE and T cells, Hu004-68-αCD3-BITE and T cells, Hu004-73-αCD3-BITE and T cells, Hu004-100-αCD3-BITE and T cells, Hu004-101-αCD3-BITE and T cells were added, respectively.
3. Overnight culture was performed at 37° C. for killing. After 24 h, the supernatant was removed and 50 μL of cell lysate (Promega, Cat. No. E1531) was added per well and incubated for 30 min at room temperature with shaking and 30 μL of Luciferase substrate (Promega, Cat. No. E151A) was added per well. On-machine detection (GloMax Navigator Microplate Luminometer, Promega, Steady-Glo protocol) was conducted.

The obtained result was shown in FIG. 3, the CD47 antibody-αCD3-BITE of clone No. 101 can well mediate the anti-lung cancer cell effect of T cells in vitro. The CD47 antibody (scFv)-αCD3-BITE of clone No. 101 had a 4-fold increase in tumor killing activity compared with the T cell control group, a 1.2-fold increase compared with clone No. 100 with low killing ability, and a 1.2-fold increase compared with the positive control antibody, which can well mediate the anti-ovarian adenocarcinoma cell effect of T cells in vitro. Together, the results of FIG. 2 and FIG. 3 demonstrated that a bispecific T-cell engager (BITE) comprising specific binding of human CD47 and human CD3 was able to well mediate T cell antitumor activity in vitro and had a broad-spectrum property.

### Example 4: Construction and Expression Verification of Recombinant Vaccinia Virus of αCD47-αCD3-BITE 4.1 Construction of pSC65 vector with αCD47-αCD3-BITE target gene

The DNA sequence of αCD47-αCD3-BITE was artificially synthesized and PCR amplification was performed using the synthesized DNA sequence as a template and using the following primers.

The primers used for amplification were:

| | αCD47-αCD3-BITE-F: |
|---|---|
| SEQ ID NO: 43 | |
| | GTACCAGGCCTAGTACTATGGAGAGGACCCTTGTCTG |
| | αCD47-αCD3-BITE-R: |
| SEQ ID NO: 44 | |
| | AATAAGCTCGAAGTCGACCTAGGAGAGATGCTGATG |

PCR reaction procedure: pre-denaturating at 94° C. for 5 min; denaturing at 98° C. for 10 seconds, 58° C.: annealing for 30 seconds, extending at 72° C. for 1 min, and reacting for 30 cycles; sufficiently extending at 72° C. for additional 10 min, ending at 25° C.

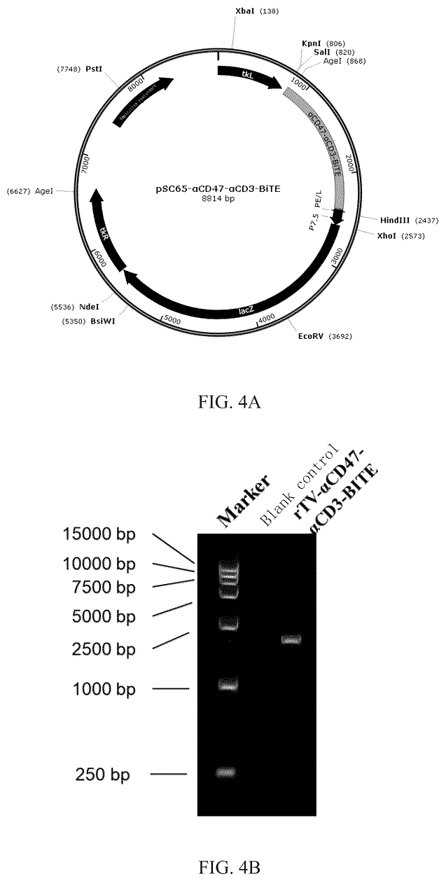

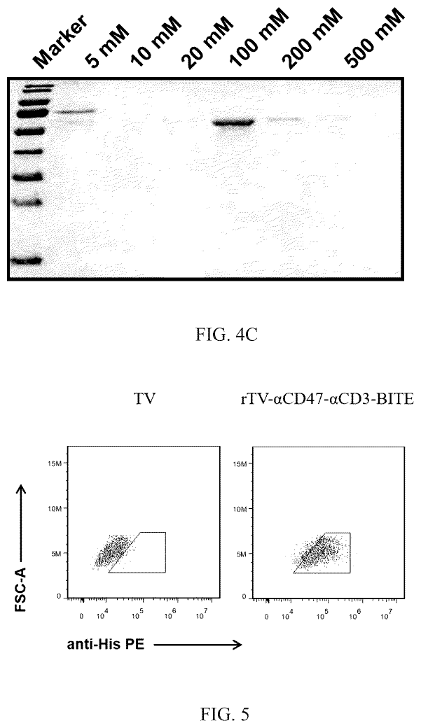

Recovery and cloning of PCR products: after amplification, the target gene was separated on a 2% agarose gel, and the pSC65 vector was linearized by Sal I digestion (Thermo Scientific, Cat. No. ER0642). The digestion gel was recovered, and the PCR fragment and vector enzyme section were recovered using a Sanprep column DNA gel recovery kit (Promega, Cat. No. A9282). The recovered gene product was linked to the digested linearized vector by homologous recombination method (Vazyme, Cat. No. c112-02). The linking product was transformed into E. coli TOP10 and grown overnight on plates containing ampicillin. On Day 2, a single colony was randomly picked for sequencing, and mutation site was corrected, and after verifying that all sequences were correct, the shuttle plasmid pSC65 of αCD47-αCD3-BITE was successfully cloned, and the plasmid construction map was shown in FIG. 4A.

### 4.2 Construction of Recombinant Vaccinia Virus αCD47-αCD3-BITE

1. Cell preparation: 143TK-cells were plated in 6-well plates at approximately 1×10⁶ cells per well. After incubated for approximately 24 h, the cells attached and spreaded across the bottom, and then the next step was proceeded.
2. Vaccinia virus incubation: 0.0125/3 PFU (PFU: plaque-forming units, virus titer)/cell of wild-type vaccinia virus Tian Tan strain was used to infect the cells. The cells were incubated in an incubator at 37° C. for 1 h and then removed. The supernatant was aspirated and washed once with 1 mL PBS and 1 mL of complete medium was added.
3. Plasmid transfection: the shuttle plasmid PSC65-αCD47-αCD3-BITE described above was transfected into 143TK-cells, and incubated at 37° C. for about 48 h, depending on the cytopathic effect.
4. 2× DMEM maintenance medium (containing 2% PS and 4% FBS) for virus plating was prepared and 2% pre-warmed low melting agarose was added followed by X-gal (final concentration 200 μg/mL).
5. The supernatant was aspirated from the 6-well plate and the mixture for plating was added to the 6-well plate at 1 mL per well. Then the mixture was carefully put into the refrigerator at 4° C. to promote solidification. After the low-melting agarose had solidified, the mixture was transferred into an incubator at 37° C. for inversion culture until clear blue spots appeared.
6. The blue spots (recombinant vaccinia virus rTV-αCD47-αCD3-BITE) were picked to 500 μL of complete medium, which was repeatedly freezing and thawing at −80° C. for more than three times to release as much virus as possible.
7. 143TK-cells were plated in 6-well plates at approximately 1×10⁶ per well. Incubation was performed for approximately 24 h until the cells adhered and spreaded across the entire floor.
8. The blue spots in the EP tube were repeatedly blown and dispersed completely.
9. The complete medium was changed to maintenance medium and then the virus solution containing the blue spots was added and incubated in an incubator at 37° C. for 3-4 h.
10. Adding screening pressure: the working concentration of BrdU was 50 μg/mL, the cells were incubated in an incubator at 37° C. for about 48 h, and plated according to the formation of virus spots. This purification process needed to be performed at least five times.
11. A small sample of recombinant vaccinia virus was then amplified, and 143TK-cells were plated in six-well plates at 1×10⁶ cells per well, using approximately 100% of the bottom area of the plate.
12. The medium in the wells was changed to 2 mL maintenance medium prior to seeding. The purified virus solution containing the blue spots was repeatedly blasted until the blue spots were dispersed. About 100 μL of virus solution was added to each well, incubated at 37° C. for about 48 h, and the samples were collected according to the formation of virus spots.
13. Collecting sample: the culture medium supernatant in the wells was carefully aspirated by 1 mL. The cells were thoroughly blown down with the remaining 1 mL of medium and taken up in EP tubes for subsequent genomic extraction and amplification as virus seed.

### 4.3 Amplification and Purification of αCD47-αCD3-BITE Recombinant Vaccinia Virus

1. VERO cell plating: 10 cm dishes, about 5×10⁶ cells per dish, it was appropriate to ensure that the cell density reached 100% when inoculating the vaccinia virus on the next day.
2. Before virus inoculation, complete medium was replaced with 8 mL maintenance medium (DMEM medium+2% FBS+1% PS), and virus was inoculated into cells in maintenance medium, with the inoculum size of about 0.02 MOI (MOI=virus PFU/cell number), which was continued to culture in an incubator containing 5% CO₂ at 37° C. for about 48 h, and the samples was collected according to the formation of virus spots.
3. Collecting vaccinia virus: 8 mL culture medium in the dish was discarded, 2 mL maintenance culture medium was taken to blow down the remaining cells, and the virus was collected into a 15 mL centrifuge tube.
4. After cryopreservation for 24 h, the obtained virus solution was repeatedly frozen and thawed twice, density gradient centrifugation was performed with 36% sucrose solution, and centrifuged at 16000 g at 4° C. for 90 min, the supernatant was carefully discarded, the virus precipitate in the centrifuge tube was dissolved with PBS buffer, dispensed and stored at −80° C., and the virus titer was to be determined.

### 4.4 Titer Determination of Recombinant Vaccinia Virus αCD47-αCD3-BITE

1. Preparation of 143TK cells: 143TK cells were plated in a 24-well plate, with about 2×10⁵ cells per well, and the cell density should reach 100% of the bottom area of the 24-well plate when used.
2. The virus was diluted, the vaccinia virus solution was diluted with maintenance medium. Starting from 1:100, 10-fold dilution was made, and the final volume was 1100 μL.
3. The complete medium in the 24-well plate was discarded, 500 μL of diluted virus solution was taken and added into the well, and two duplicate wells were made. Incubation was performed at 37° C. in a 5% CO₂ incubator for about 48 h, and the plating time was determined according to the formation of virus plaque.
4. Plating method: 8 mL of plating medium containing 2× DMEM medium+4% FBS+2% PS and 8 mL of low-melting agarose thawed in a boiling water bath and placed in 37° C. water bath were prepared, the two were mixed, and then X-gal was added to the mixture, with the final concentration of 200 μg/mL, for later use.
5. The supernatant was aspirated from the 24-well plate. The plating mixture in step 4 was immediately added to a 24-well plate at 500 μL per well. Then it was carefully put into a refrigerator at 4° C. to promote solidification, and after the low-melting-point agarose had solidified, the same was transferred into an incubator at 37° C. for inversion culture until clear blue spots appeared.
6. Virus plaque counting: first, whether the number of viral plaques decreases in a ten-fold trend was observed, then the number of blue spots with only one digit in two duplicate wells of seed virus was counted. The sum of the values of blue spots obtained in the two wells multiplied the reciprocal value of the corresponding dilution of the well to obtain the virus titer in 1 mL.
   4.5 Verification of Expression of Recombinant Vaccinia Virus rTV-αCD47-αCD3-BITE
1. Collecting virus supernatant: 10 cm culture dish was taken, and 5×10⁶ VERO cells/dish were inoculated, so as to ensure that the cell density reached 100% when inoculating the vaccinia virus on the next day. Before virus inoculation, complete medium was replaced with 8 mL maintenance medium (DMEM medium+2% FBS+1% PS); then viruses were added, with the inoculum size of about 0.02 MOI (MOI=virus PFU/cell number), incubated in a 37° C. 5% CO₂ incubator for 48 h; the cell supernatant was collected according to the formation of virus plaque, centrifuged at 10000 g for 5 min, and the supernatant was taken into a new centrifuge tube.
2. Subjecting the virus supernatant to His tag protein purification: the purification was performed manually using a syringe and the packed nickel beads were Ni-NTA Agarose (QIAGEN, Cat. No. 30210); after the completion of column loading, non-specifically adsorbed proteins were eluted with 5 nM and 10 nM imidazole (dissolved in 50 mM NaH₂PO₄: 2H₂O, pH 8.0, 300 mM NaCl) at the natural flow rate, and the filtrate was collected; the target protein was then eluted with 20 nM, 100 nM, 200 nM, 500 nM imidazole (dissolved in 50 mM NaH₂PO₄: 2H₂O, pH 8.0, 300 mM NaCl) and the corresponding filtrate was collected.
3. 30 μL of the filtrate was taken to prepare samples, 10% SDS-PAGE detection was performed and the results were shown in FIG. 4B. Samples were loaded sequentially from 5 mM imidazole eluate to 500 mM imidazole eluate. As shown in FIG. 4C, the protein of interest appeared in the 200 nM eluate, with a band size of around 54 kD.

### Example 5: Affinity Detection of Recombinant Vaccinia Virus rTV-αCD47-αCD3-BITE

1. Collecting virus supernatant: 10 cm culture dish was taken, and 5×10⁶ VERO cells/dish were inoculated, so as to ensure that the cell density reached 100% when inoculating the vaccinia virus on the next day. Before virus inoculation, the complete medium was replaced with 8 mL maintenance medium (DMEM medium+2% FBS+1% PS); then virus was added, with the inoculation amount of about 0.02 MOI (MOI=virus PFU/cell number), and at the same time, the wild-type virus control was set; incubation was performed in a 37° C. 5% CO₂ incubator for 48 h; the cell supernatant was collected according to the formation of virus plaque, centrifuged at 10000 g for 5 min, and the supernatant was taken into a new centrifuge tube for His tag protein purification.
2. His tag protein purification was performed by the method as described above.
3. Preparation of CD47-A549 cells: 2×10⁶ cells were divided into two EP tubes, centrifuged at 800 g for 3 min and the supernatant was discarded.
4. Wash was conducted twice with 1 mL of pre-chilled wash (1×PBS+2% FBS), centrifugation was performed at 800 g for 3 min and the supernatant was discarded.
5. An appropriate amount of purified protein was incubated with CD47-A549 cells at room temperature for 15 min.
6. Wash was conducted twice with 1 mL of pre-chilled wash, centrifugation was performed at 800 g for 3 min and the supernatant was discarded.
7. 0.1 μL of PE-labeled anti-His antibody was added to each sample for 15 min at room temperature.
8. Wash was conducted twice with 1 mL of pre-chilled wash, centrifugation was performed at 800 g for 3 min and the supernatant was discarded.
9. Each sample was resuspended in 200 μL of wash solution and affinity was determined by flow cytometry.

The result in FIG. 5 showed that cell supernatant expressed by vaccinia virus recombined with αCD47-αCD3-BITE can bind to CD47 with a positive rate of 85.9% compared to wild-type vaccinia virus. The positive clone was picked for the passage stability test, and the virus strain with strong passage stability and high expression of the target protein was selected and deposited, which was named rTV-αCD47-αCD3-BITE, with the deposit accession number: CCTCC NO: V202081, and deposited on Jan. 2, 2021 at China Center for Type Culture Collection, address: Wuhan University, Wuhan, China.

### Example 6: Effect of Recombinant Vaccinia Virus with αCD47-αCD3-BITE In Vitro Against Lung Cancer

1. 143TK-Cell Plating: 143TK-cells were plated in a 6-well plate, with about 1×10⁶ cells per well, and the cell density should reach 100% of the bottom area of the 6-well plate when used.
2. The complete medium was changed to 2 mL maintenance medium per well (DMEM medium+2% FBS+1% PS) prior to virus inoculation and the cells were inoculated with the virus at 0.02 MOI. Cells were incubated at 37° C. in a 5% CO₂ incubator for about 48 h, and when to collect the supernatant was determined according to the virus plaque formation. At the same time, wild vaccinia virus was used as control.
3. 100 μL of the supernatant was taken separately for subsequent A549 in vitro killing assay.
4. The in vitro killing assay of A549 cells was performed according to the instructions of kit (Dojindo, Cat. No. CK17); 100 μL of resuspended A549 cells was pipetted into a 96-well plate with 1×10⁴ cells per well, and incubated at 37° C., 5% CO₂ overnight.
5. The next day, the supernatant was added, cells were incubated at 37° C., 5% CO₂ for 1 h, and then T cells were added at an effect-target ratio of 5:1, and cells were continued to be incubated for 4 h at 37° C., 5% CO₂.
6. Following the addition of 20 μL of lysis buffer to the high control wells, 20 μL of the medium was added to the low control wells and background wells, and cells were incubated for 30 min at 37° C., 5% CO₂.
7. 100 μL of supernatant was pipetted from each well into a new 96-well plate.
8. After adding 100 μL of the developing solution to each well, it was reacted for 5 min at room temperature in the dark.
9. Finally, the assay was stopped by adding 50 μL stop solution to each well, the absorbance at 490 nm was immediately measured with a microplate reader.

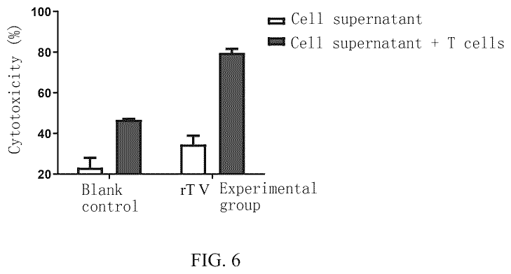

Results were as shown in FIG. 6, recombinant vaccinia virus loaded with αCD47-αCD3-BITE significantly increased the killing of A549 cells in vitro by as much as 4-fold compared to the blank control group.

In conclusion, αCD47-αCD3-BITE protein combined with T cells had very strong killing activity on tumor cells, and the recombinant vaccinia virus rTV-αCD47-αCD3-BITE prepared as oncolytic virus can also significantly control a variety of solid tumors such as human lung cancer, which had very high application value for the treatment of tumors and was simple to prepare, thereby being convenient for large-scale preparation and promotion.

### Example 7: In Vitro Anti-Human Ovarian Adenocarcinoma Cell Effect of T Cells Mediated by αCD47-αCD3-BITE of Different Structural Formulae

In order to prove the anticancer effect of αCD47-αCD3-BITE proteins with different structures, the applicant respectively designed the following BITE proteins:

BITE1:VL_{αCD47}-L-VH_{αCD3}-L-VL_{αCD3}-L-VH_{αCD47}

the VH_{αCD47} was as shown in SEQ ID NO: 19, the VL_{αCD47} was as shown in SEQ ID NO: 21, the VH_{αCD3} was as shown in SEQ ID NO: 25, the VL_{αCD3} was as shown in SEQ ID NO: 26, and the L was GGGGSGGGGSGGGGS.

BITE2:VH_{αCD47}-L-VH_{αCD3}-L-VL_{αCD3}-L-VL_{αCD47}

the VH_{αCD47} was as shown in SEQ ID NO: 19, the VL_{αCD47} was as shown in SEQ ID NO: 21, the VH_{αCD3} was as shown in SEQ ID NO: 25, the VL_{αCD3} was as shown in SEQ ID NO: 26, and the L was GGGGSGGGGSGGGGS.

BITE3:VH_{αCD47}-L-VL_{αCD47}-L-VH_{αCD3}-L-VL_{αCD3}

the VH_{αCD47} was as shown in SEQ ID NO: 19, the VL_{αCD47} was as shown in SEQ ID NO: 21, the VH_{αCD3} was as shown in SEQ ID NO: 25, the VL_{αCD3} was as shown in SEQ ID NO: 26; one linker peptide was GGGGSGGGGSGGGGS.

BITE4: Like BITE1, only the linker peptide was replaced with GGGGGG;

BITE5: Like BITE1, the VH_{αCD3} was as shown in SEQ ID NO: 27 and VL_{αCD3} was as shown in SEQ ID NO: 28.

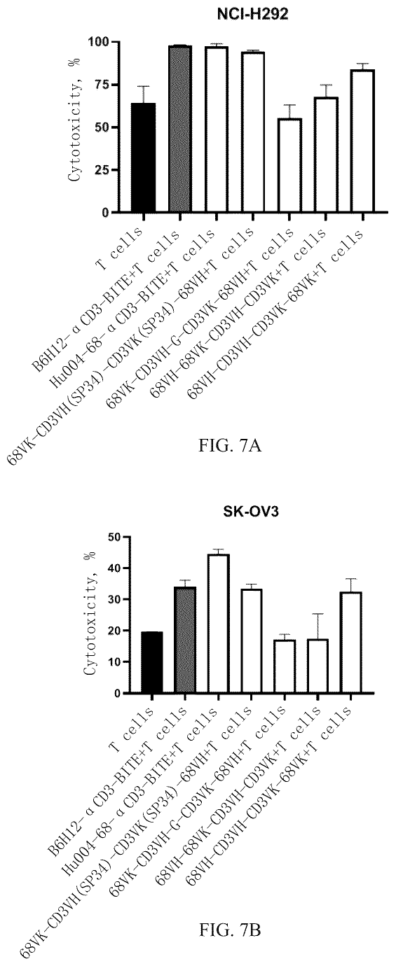

The example was the same as Examples 2 and 3 and the results were shown in FIG. 7.

VL_{αCD47}-L-VH_{αCD3}-L-VL_{αCD3}-L-VH_{αCD47}, in which L was (G4S) 3, was the optimal design of αCD47-αCD3-BITE, which can mediate strong killing function against tumor cells; when the antibody clone types of CD47 or CD3 (such as clone No. SP34) was changed, the BiTE can still have strong killing function; VH_{αCD47}-L-VH_{αCD3}-L-VL_{αCD3}-L-VL_{αCD47}, where L was (G4S) 3, was a suboptimal design for αCD47-αCD3-BITE, but still had tumor killing activity similar to the control antibody.

The above-described embodiments are exemplary and are not to be construed as limiting the present invention, and variations, modifications, substitutions, and alterations to the above-described embodiments may be made by those of ordinary skill in the art within the scope of the present invention.

1. An αCD47 and αCD3 bispecific T-cell engager αCD47-αCD3 BiTE, comprising a fusion protein of any one of the following formulae:
VL_{αCD47}-L-VH_{αCD3}-L-VL_{αCD3}-L-VH_{αCD47};
or
VH_{αCD47}-L-VH_{αCD3}-L-VL_{αCD3}-L-VL_{αCD47}
wherein L represents a linker peptide;
wherein the VH_{αCD47} is the heavy chain variable region of a CD47 antibody comprising the following three complementary determining regions:
(i) VH CDR1 consisting of the following sequence: SEQ ID NO: 1, or a sequence having one or several amino acid substitutions, deletions, or additions compared thereto,
(ii) VH CDR2 consisting of the following sequence: SEQ ID NO: 2, or a sequence having one or several amino acid substitutions, deletions, or additions compared thereto, and
(iii) VH CDR3 consisting of the following sequence: SEQ ID NO: 3, or a sequence having one or several amino acid substitutions, deletions, or additions compared thereto;
preferably, the substitution of any one of (i)-(iii) is a conservative substitution;
preferably, the VH_{αCD47} comprises VH CDR1 as shown in SEQ ID NO: 1, VH CDR2 as shown in SEQ ID NO: 2, VH CDR3 as shown in SEQ ID NO: 3;
wherein the VL_{αCD47} is the light chain variable region of a CD47 antibody comprising the following three complementary determining regions:
(iv) VL CDR1 consisting of the following sequence: a sequence as shown in any one of SEQ ID NO: 4, SEQ ID NO: 7, SEQ ID NO: 10, SEQ ID NO: 13, or SEQ ID NO: 16, or having one or several amino acid substitutions, deletions, or additions compared thereto,
(v) VL CDR2 consisting of the following sequence: a sequence as shown in any one of SEQ ID NO: 5, SEQ ID NO: 8, SEQ ID NO: 11, SEQ ID NO: 14, or SEQ ID NO: 17, or having one or several amino acid substitutions, deletions, or additions compared thereto, and
(vi) VL CDR3 consisting of the following sequence: a sequence as shown in any one of SEQ ID NO: 6, SEQ ID NO: 9, SEQ ID NO: 12, SEQ ID NO: 15, or SEQ ID NO: 18, or having one or several amino acid substitutions, deletions, or additions compared thereto;
preferably, the substitution of any one of (iv)-(vi) is a conservative substitution;
preferably, the VL_{αCD47} comprises VL CDR1 as shown in SEQ ID NO: 4, VL CDR2 as shown in SEQ ID NO: 5, and VL CDR3 as shown in SEQ ID NO: 6;
or the VL_{αCD47} comprises VL CDR1 as shown in SEQ ID NO: 7, VL CDR2 as shown in SEQ ID NO: 8, and VL CDR3 as shown in SEQ ID NO: 9;
or the VL_{αCD47} comprises VL CDR1 as shown in SEQ ID NO: 10, VL CDR2 as shown in SEQ ID NO: 11, and VL CDR3 as shown in SEQ ID NO: 12;
or the VL_{αCD47} comprises VL CDR1 as shown in SEQ ID NO: 13, VL CDR2 as shown in SEQ ID NO: 14, and VL CDR3 as shown in SEQ ID NO: 15;
or the VL_{αCD47} comprises VL CDR1 as shown in SEQ ID NO: 16, VL CDR2 as shown in SEQ ID NO: 17, and VL CDR3 as shown in SEQ ID NO: 18.

2. The bispecific T-cell engager of claim 1, wherein the VH_{αCD47} comprises three CDRs contained in the heavy chain variable region as shown in SEQ ID NO: 19;
preferably, the VL_{αCD47} comprises three CDRs contained in the light chain variable region as shown in any one of SEQ ID NOs: 20-24;
preferably, the three CDRs contained in the heavy chain variable region and/or the three CDRs contained in the light chain variable region are defined by the Kabat, Chothia, or IMGT numbering system.

3. The bispecific T-cell engager of claim 1, wherein the VH_{αCD47} comprises an amino acid sequence selected from the group consisting of:
(i) a sequence as shown in SEQ ID NO: 19;
(ii) a sequence having one or several amino acid substitutions, deletions, or additions compared to the sequence as shown in SEQ ID NO: 19; or
(iii) a sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as shown in SEQ ID NO: 19;
and/or,
the VL_{αCD47} comprises an amino acid sequence selected from the group consisting of:
(iv) a sequence as shown in any one of SEQ ID NOs: 20-24;
(v) a sequence having one or several amino acid substitutions, deletions, or additions compared to the sequence as shown in any one of SEQ ID NOs: 20-24; or
(vi) a sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as shown in any one of SEQ ID NOs: 20-24;
preferably, the substitution in (ii) or (v) is a conservative substitution.

4. The bispecific T-cell engager of claim 1, wherein the VH_{αCD3} comprises an amino acid sequence selected from the group consisting of:
(i) a sequence as shown in SEQ ID NO: 25 or 27;
(ii) a sequence having one or several amino acid substitutions, deletions, or additions compared to the sequence as shown in SEQ ID NO: 25 or 27; or
(iii) a sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as shown in SEQ ID NO: 25 or 27;
and/or,
the VL_{αCD3} comprises an amino acid sequence selected from the group consisting of:
(iv) a sequence as shown in SEQ ID NO: 26 or 28;
(v) a sequence having one or several amino acid substitutions, deletions, or additions compared to the sequence as shown in SEQ ID NO: 26 or 28; or
(vi) a sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as shown in SEQ ID NO: 26 or 28;
preferably, the substitution in (ii) or (v) is a conservative substitution.

5. The bispecific T-cell engager of claim 1, wherein the VH_{αCD47}, VH_{αCD3}, VL_{αCD47}, and VL_{αCD3} are linked by a linker peptide;
preferably, the VH_{αCD47}, VH_{αCD3}, VL_{αCD47}, and VL_{αCD3} are linked by one, two, or three linker peptides;
more preferably, L is KESGSVSSEQLAQFRSLD (SEQ ID NO: 45), EGKSSGSGSESKST (SEQ ID NO: 46), GGGGGG (SEQ ID NO: 47), GGGGGGGG (SEQ ID NO: 48) or (GGGGS) n (SEQ ID NO: 49); further preferably, n is an integer from 1 to 5, most preferably n is 3;
preferably, the bispecific T-cell engager comprises a fusion protein of the following formula:
VH_{αCD47}-L-VH_{αCD3}-L-VL_{αCD3}-L-VL_{αCD47}
wherein the VH_{αCD47} is as shown in SEQ ID NO: 19, the VL_{αCD47} is as shown in SEQ ID NO: 21 or 24, the VH_{αCD3} is as shown in SEQ ID NO: 25, VL_{αCD3} is as shown in SEQ ID NO:
26, and the L is GGGGSGGGGSGGGGS (SEQ ID NO: 50).

6. (canceled)

7. An expression framework for the bispecific T-cell engager of claim 1:
5′-E1-E2-E3-E4-E5-E6-E7-3′
wherein:
E1 is a CMV enhancer or/and other cis-acting elements, preferably comprises the nucleic acid sequence as shown in SEQ ID NO: 37;
E2 is a recombinantly expressed promoter, preferably a CMV promoter, more preferably comprises the nucleic acid sequence as shown in SEQ ID NO: 38;
E3 is a 5′ untranslated region, optionally comprises no or one intron sequence, optionally comprises one or more restriction enzyme sites, preferably comprises the nucleic acid sequence as shown in SEQ ID NO: 39;
E4 is a coding nucleotide sequence of the signal peptide of the BiTE protein; preferably, the signal peptide is derived from a human or mouse signal peptide; preferably E4 comprises the nucleic acid sequence as shown in SEQ ID NO: 40;
E5 is a coding nucleotide sequence of the bispecific T-cell engager of claim 1;
E6 is a 3′ untranslated region, optionally comprises one or more restriction enzyme sites, preferably comprises the nucleic acid sequence as shown in SEQ ID NO: 41;
E7 is an SV40 transcription termination signal region, preferably comprises the nucleic acid sequence as shown in SEQ ID NO: 42.

8. A recombinant oncolytic virus, the recombinant oncolytic virus being operably inserted with or comprising the expression framework of claim 7; wherein
preferably, the expression framework is located in the thymidine kinase (TK) region of the recombinant oncolytic virus;
preferably, the expression framework can be expressed alone, or be fusion expressed with other genes or fragments;
preferably, the recombinant oncolytic virus further comprises gene coding sequences for other immunomodulatory factors, more preferably, the other immunomodulatory factors include but are not limited to IL-1, IL-2, IL-3, IL-7, IL-11, IL-12, IL-15, IL-17, IL-18, IL-21, IL-33, IL-35, IL-37, GM-CSF, IFN-α, IFN-β, IFN-γ, anti-PD-1/PD-L1 antibody, anti-CTLA-4 antibody, anti-Lag-3 antibody, anti-TIGIT antibody, or anti-Tim-3 antibody; or
the recombinant oncolytic virus further comprises a gene coding sequence of an apoptosis- and pyroptosis-related protein, preferably the apoptosis- and pyroptosis-related protein is selected from apoptosis-related factor 1, interleukin-1 β converting enzyme, Bcl-2 protein, Fas/APO-1, p53, myc, ataxia telangiectasia mutant gene, gasdermin D, or gasdermin E; or
the recombinant oncolytic virus further comprises a small RNA targeting immunomodulatory genes, apoptosis and pyroptosis genes.

9. The recombinant oncolytic virus of claim 8, wherein the viral backbone of the oncolytic virus is derived from a modified or engineered vaccinia virus Tian Tan strain, vaccinia virus New York strain, vaccinia virus Copenhagen strain, vaccinia virus canary strain, vaccinia virus Ankara strain, adenovirus, adeno-associated virus, herpes simplex virus, varicella-zoster virus, respiratory syncytial virus, Semliki forest virus, EB virus, cytomegalovirus, human Herpesvirus type 6, smallpox virus, vaccinia virus, molluscum contagiosum virus, sheep aphthovirus, reovirus, rotavirus, enterovirus, Seneca virus, poliovirus, coxsackie virus, rhinovirus, hepatitis A virus, foot and mouth disease virus, togavirus, alphavirus, Semiliki forest virus, eastern equine encephalitis virus, Sindbis virus, rubella virus, coronavirus, flavivirus, hepatitis C virus, Japanese encephalitis virus, St. Louis encephalitis virus, Murray Valley fever virus, yellow fever virus, West Nile virus, zika virus, dengue virus, Ebola virus, Marburg virus, arenavirus, Lassa fever virus, lymphocytic choriomeningitis virus, Pichinde virus, Junin virus, Machupo virus, Hantavirus, Rift Valley fever virus, Paramyxovirus, human parainfluenza virus, Mumps virus, simian virus 5, measles virus, vesicular stomatitis virus, rabies virus, orthomyxovirus, influenza A virus, influenza B virus, influenza C virus, hepatitis D virus, simian immunodeficiency virus, human immunodeficiency virus type 1 and human immunodeficiency virus type 2, Rous sarcoma virus, human T-cell leukemia virus type 1, simian foamy virus, hepatitis B virus, hepatitis E virus, human papilloma virus, or polyomavirus;
preferably, the oncolytic viral backbone is an intracellular maturation virus, an intracellular packaging virus, a cell-associated packaging virus, or an extracellular packaging virus.

10. A recombinant vaccinia virus Tian Tan strain rTV-αCD47-αCD3-BITE, with the deposit accession number of CCTCC NO: V202081.

11. A method for preparing the recombinant oncolytic virus of claim 8, comprising the steps of:
1. synthesizing an expression framework of the bispecific T-cell engager BiTE of claim 7;
2. subcloning the expression framework obtained in step 1) into a shuttle plasmid of an oncolytic virus to construct a recombinant plasmid vector;
3. transfecting the recombinant plasmid vector obtained in step 2) into an oncolytic virus, and screening the same so as to obtain a recombinant oncolytic virus;
optionally, culturing the obtained recombinant oncolytic virus;
preferably, the method comprising the steps of:
1. synthesizing an expression framework of the bispecific T-cell engager αCD47-αCD3-BITE, which comprises a nucleic acid sequence as shown in any one of SEQ ID Nos: 31-42;
2. subcloning the synthesized expression framework into the TK region of the vaccinia virus shuttle plasmid (pSC65) to construct the recombinant plasmid pSC65-αCD47-αCD3-BITE;
3. transfecting the pSC65-αCD47-αCD3-BITE plasmid into TK143-cells which have been infected with wild-type vaccinia virus by means of gene homologous recombination, and homologously recombing the two to produce recombinant vaccinia virus rTV-αCD47-αCD3-BITE; screening the same to obtain a recombinant oncolytic vaccinia virus wherein the TK region comprises a coding sequence of αCD47-αCD3-BITE;
preferably, the expression framework of the bispecific T-cell engager αCD47-αCD3-BITE is controlled by the early/late promoter p7.5 of vaccinia virus.

12. (canceled)

13. A method of treating a tumor, comprising administering to a subject in need thereof a therapeutically effective amount of the bispecific T-cell engager of claim 1 or the recombinant oncolytic virus of claim 8;
preferably, the tumor being selected from the group consisting of B-cell lymphoma; T-cell lymphoma; melanoma; prostate cancer; renal cell carcinoma; sarcoma; glioma, such as high-grade glioma; blastoma, such as neuroblastoma; osteosarcoma; plasmacytoma; histiocytoma; pancreatic cancer; breast cancer; lung cancer, such as small cell lung cancer and non-small cell lung cancer; gastric cancer; liver cancer; colon cancer; rectal cancer; esophageal cancer; colorectal cancer; hematopoietic cancer; testicular cancer; cervical cancer; ovarian cancer; bladder cancer; squamous cell cancer; adenocarcinoma; AIDS-related lymphoma; bladder cancer; brain cancer; nervous system cancer; head and neck cancer; head and neck squamous cell carcinoma; Hodgkin's lymphoma; non-Hodgkin's lymphoma; or hematological tumorigenesis disease.


SEQUENCE LISTING (50 sequences)

SEQ ID NO: 1 - protein, 5 aa, Artificial Sequence
  Heavy chain variable region complementarity determining region 1 (VH CDR1)
GYGMS

SEQ ID NO: 2 - protein, 17 aa, Artificial Sequence
  Heavy chain variable region complementarity determining region 2 (VH CDR2)
TITSGGTYTYYPDSVKG

SEQ ID NO: 3 - protein, 9 aa, Artificial Sequence
  Heavy chain variable region complementarity determining region 3 (VH CDR3)
SLAGNAMDY

SEQ ID NO: 4 - protein, 11 aa, Artificial Sequence
  Light chain variable region complementarity determining region 1 (VL CDR1)
RASQSISSWLA

SEQ ID NO: 5 - protein, 8 aa, Artificial Sequence
  Light chain variable region complementarity determining region 2 (VL CDR2)
KASSLESG

SEQ ID NO: 6 - protein, 5 aa, Artificial Sequence
  Light chain variable region complementarity determining region 3 (VL CDR3)
QHRRA

SEQ ID NO: 7 - protein, 11 aa, Artificial Sequence
  Light chain variable region complementarity determining region 1 (VL CDR1)
RASQGVTSNLA

SEQ ID NO: 8 - protein, 7 aa, Artificial Sequence
  Light chain variable region complementarity determining region 2 (VL CDR2)
GASTRAT

SEQ ID NO: 9 - protein, 5 aa, Artificial Sequence
  Light chain variable region complementarity determining region 3 (VL CDR3)
QHRGT

SEQ ID NO: 10 - protein, 11 aa, Artificial Sequence
  Light chain variable region complementarity determining region 1 (VL CDR1)
RASQGISSRLA

SEQ ID NO: 11 - protein, 7 aa, Artificial Sequence
  Light chain variable region complementarity determining region 2 (VL CDR2)
AASSLQS

SEQ ID NO: 12 - protein, 5 aa, Artificial Sequence
  Light chain variable region complementarity determining region 3 (VL CDR3)
QQANS

SEQ ID NO: 13 - protein, 11 aa, Artificial Sequence
  Light chain variable region complementarity determining region 1 (VL CDR1)
RASQSISNWVA

SEQ ID NO: 14 - protein, 7 aa, Artificial Sequence
  Light chain variable region complementarity determining region 2 (VL CDR2)
KASTLAN

SEQ ID NO: 15 - protein, 5 aa, Artificial Sequence
  Light chain variable region complementarity determining region 3 (VL CDR3)
QHRVT

SEQ ID NO: 16 - protein, 11 aa, Artificial Sequence
  Light chain variable region complementarity determining region 1 (VL CDR1)
RASQSISNWLA

SEQ ID NO: 17 - protein, 7 aa, Artificial Sequence
  Light chain variable region complementarity determining region 2 (VL CDR2)
KASNLES

SEQ ID NO: 18 - protein, 5 aa, Artificial Sequence
  Light chain variable region complementarity determining region 3 (VL CDR3)
QQFGT

SEQ ID NO: 19 - protein, 118 aa, Artificial Sequence
  Heavy chain variable region of CD47 antibody (VHalphaCD47)
EVQLVESGGGLVQPGGSLRLSCAASGFTFSGYGMSWVRQAPGKGLEWVATITSGGTYTYYPDSVKGRFTISRDNAKNSLYLQMNSLRAEDTAVYYCARSLAGNAMDYWGQGTLVTVSS

SEQ ID NO: 20 - protein, 103 aa, Artificial Sequence
  Light chain variable region of CD47 antibody (VLalphaCD47)
DIQMTQSPSTLSASVGDRVTITCRASQSISSWLAWYQQKPGKAPKLLIYKASSLESGVPSRFSGSGSGTEFTLTISSLQPDDFATYYCQHRRAFGQGTKVEIK

SEQ ID NO: 21 - protein, 103 aa, Artificial Sequence
  Light chain variable region of CD47 antibody (VLalphaCD47)
EIVLTQSPVTLSVSPGERATLSCRASQGVTSNLAWYQQKPGQAPRLLIYGASTRATGVPARFSGSGSGTEFTLTISSLQSEDFAVYYCQHRGTFGQGTKVDIK

SEQ ID NO: 22 - protein, 103 aa, Artificial Sequence
  Light chain variable region of CD47 antibody (VLalphaCD47)
DIQMTQSPSSVSASVGDRVTITCRASQGISSRLAWYQQKPGKAPKLLIYAASSLQSGVPSRFSGSGSGTDFTLTISSLQPEDFATYYCQQANSFGGGTKVEIK

SEQ ID NO: 23 - protein, 103 aa, Artificial Sequence
  Light chain variable region of CD47 antibody (VLalphaCD47)
DIQMTQSPTTLSASVGHRVTINCRASQSISNWVAWYQQKAGSAPKLLIYKASTLANGVPSRFSGSGSGTEFILTISSLQPEDVATYYCQHRVTFGPGTKVDLK

SEQ ID NO: 24 - protein, 103 aa, Artificial Sequence
  Light chain variable region of CD47 antibody (VLalphaCD47)
DIQMTQSPSTLSASVGDRVTITCRASQSISNWLAWYQQKPGEAPKLLIYKASNLESGVPSRFSGSGSGTEFTLTISSLQPDDFATYYCQQFGTFGQGTKVEIK

SEQ ID NO: 25 - protein, 119 aa, Artificial Sequence
  Heavy chain variable region of CD3 antibody (VHalphaCD3)
QVQLVQSGAEVKKPGASVKVSCKASGYTFTRYTMHWVRQAPGQGLEWMGYINPSRGYTNYNQKFKDRVTITADKSTSTAYMELSSLRSEDTAVYYCARYYDDHYCLDYWGQGTTVTVSS

SEQ ID NO: 26 - protein, 106 aa, Artificial Sequence
  Light chain variable region of CD3 antibody (VLalphaCD3)
EIVLTQSPATLSLSPGERATLSCSASSSVSYMNWYQQKPGKAPKRLIYDTSKLASGVPSRFSGSGSGTDFTLTINSLEAEDAATYYCQQWSSNPFTFGQGTKVEIK

SEQ ID NO: 27 - protein, 122 aa, Artificial Sequence
  Heavy chain variable region of CD3 antibody (VHalphaCD3)
EVQLVESGGGLVQPKGSLKLSCAASGFTFNTYAMNWVRQAPGKGLEWVARSKYNNYATYYADSVKDRFTISRDDSQSILYLQMNNLKTEDTAMYYCVRHGNFNSYVSWFAYWGQGTLVTVSS

SEQ ID NO: 28 - protein, 110 aa, Artificial Sequence
  Light chain variable region of CD3 antibody (VLalphaCD3)
GSQAVVTQESALTTSPGETVTLTCRSSTGAVTTSNYANWVQEKPDHLFTGLIGGTNKRAPGVPARFSGSLIGKAALTITGAQTEDEAIYFCALWYSNLWVFGGGTKLTVL

SEQ ID NO: 29 - protein, 118 aa, Artificial Sequence; identical to SEQ ID NO: 19
  Heavy chain variable region of B6H12

SEQ ID NO: 30 - protein, 107 aa, Artificial Sequence
  Light chain variable region of B6H12
EIVLTQSPATLSLSPGERATLSCRASQTISDYLHWYQQKPGQAPRLLIKFASQSISGIPARFSGSGSGTDFTLTISSLEPEDFAVYYCQNGHGFPRTFGQGTKVEIK

SEQ ID NO: 31 - DNA, 309 bp, Artificial Sequence
  Light chain variable region of CD47 antibody (VLalphaCD47)
gacattcagatgacacagtctccatctacactgtccgcctccgtgggcgaccgggtgaccattacatgccgcgcctctcagtctattagctcttggctcgcctggtatcagcagaagcccggcaaggcccctaagctgctgatttacaaggcctccagtctggaatctggcgtgccatctaggttcagcggctctggcagcggcaccgagttcacactcacaatttcttccttacagcccgacgacttcgccacatactactgccagcaccggagagccttcggccagggcacaaaggtggagatcaag

SEQ ID NO: 32 - DNA, 309 bp, Artificial Sequence
  Light chain variable region of CD47 antibody (VLalphaCD47)
gagatcgtgctgacccagtctccagtgacactgagcgtgtcccctggcgagagagccaccctgtcttgcagagcctctcagggcgtgacatctaacctcgcctggtatcagcagaagcctggccaggccccaagactcctgatttacggcgcctctacacgcgccacaggcgtgcccgcccggttcagcggctctggctccggcaccgagttcacactcacaattagctctcttcagtccgaggacttcgccgtgtactactgccagcaccgcggcacattcggccagggcacaaaggtggacattaag

SEQ ID NO: 33 - DNA, 309 bp, Artificial Sequence
  Light chain variable region of CD47 antibody (VLalphaCD47)
gacattcagatgacacagtccccatctagcgtgtctgcctccgtgggcgaccgggtgacaattacatgccgcgcctctcagggcattagctcacggctcgcctggtatcagcagaagcctggcaaggcccctaagctgctgatttacgccgcctcttccctccagagcggcgtgccatctaggttctctggctccggctctggcaccgacttcacactgaccatttcttcccttcaacccgaggacttcgccacatactactgccagcaggccaactccttcggcggcggcacaaaggtggagatcaag

SEQ ID NO: 34 - DNA, 309 bp, Artificial Sequence
  Light chain variable region of CD47 antibody (VLalphaCD47)
gacatccagatgacacagtctcctacaacactgagcgcctccgtgggccacagagtgacaattaactgccgcgcctctcagtctatctctaactgggtggcctggtatcagcagaaggccggctccgccccaaagctgctgatttacaaggcctctacactcgccaacggcgtgcctagccggttcagcggctccggctctggcaccgagttcattctcaccatttcttccttgcaacccgaggacgtggccacatactactgccagcacagggtgaccttcggcccaggcaccaaggtggacctcaag

SEQ ID NO: 35 - DNA, 309 bp, Artificial Sequence
  Light chain variable region of CD47 antibody (VLalphaCD47)
gacattcagatgacacagtccccatccaccctgagcgcctccgtgggcgacagagtgaccattacatgccgcgcctctcagtctatttccaactggctcgcctggtatcagcagaagccaggcgaggcccctaagctgctgatttacaaggcctctaacttggagtctggcgtgccatctaggttctccggcagcggctccggcaccgagttcacactcacaattagctctttacaacccgacgacttcgccacatactactgccagcagttcggcaccttcggccagggcacaaaggtggagatcaag

SEQ ID NO: 36 - DNA, 354 bp, Artificial Sequence
  Heavy chain variable region of CD47 antibody (VHalphaCD47)
gaggtgcagctggtggagtctggcggcggcctcgtgcagcctggcggctccctgagactgtcctgcgccgcctctggcttcacattctccggctacggcatgtcttgggtgagacaggccccaggcaagggcctagagtgggtggccacaattacatccggcggcacatacacctactaccccgacagcgtgaagggcagattcaccatttcccgcgacaacgccaagaactctctgtacctccagatgaacagcctgagagccgaggacaccgccgtgtactactgcgcccggtcactcgccggcaacgctatggactactggggccagggcacactcgtgaccgtgtctagc

SEQ ID NO: 37 - DNA, 380 bp, Artificial Sequence
  CMV enhancer or/and other cis-acting elements (E1)
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 38 - DNA, 204 bp, Artificial Sequence
  Promoter for recombinant expression (E2)
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 39 - DNA, 331 bp, Artificial Sequence
  5′ untranslated region (E3)
cgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctatcgattgaattccacc

SEQ ID NO: 40 - DNA, 45 bp, Artificial Sequence
  Coding nucleotide sequence of signal peptide of BiTE protein (E4)
atgggatggtcatgtatcatcctttttctagtagcaactgcaacc

SEQ ID NO: 41 - DNA, 20 bp, Artificial Sequence
  3′ untranslated region (E6)
aagcttggccgccatggccc

SEQ ID NO: 42 - DNA, 135 bp, Artificial Sequence
  SV40 transcription termination signal region (E7)
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 43 - DNA, 37 bp, Artificial Sequence
  alphaCD47-alphaCD3-BITE-F
gtaccaggcctagtactatggagaggacccttgtctg

SEQ ID NO: 44 - DNA, 36 bp, Artificial Sequence
  alphaCD47-alphaCD3-BITE-R
aataagctcgaagtcgacctaggagagatgctgatg

SEQ ID NO: 45 - protein, 18 aa, Artificial Sequence
  linker peptide
KESGSVSSEQLAQFRSLD

SEQ ID NO: 46 - protein, 14 aa, Artificial Sequence
  linker peptide
EGKSSGSGSESKST

SEQ ID NO: 47 - protein, 6 aa, Artificial Sequence
  linker peptide
GGGGGG

SEQ ID NO: 48 - protein, 8 aa, Artificial Sequence
  linker peptide
GGGGGGGG

SEQ ID NO: 49 - protein, 5 aa, Artificial Sequence
  linker peptide
GGGGS

SEQ ID NO: 50 - protein, 15 aa, Artificial Sequence
  linker peptide
GGGGSGGGGSGGGGS